(19) United States
(12) Patent Application Publication
(10) Pub. No.: US 2017/0102392 A1
(43) Pub. Date: Apr. 13, 2017

(54) Early Diagnosis of Autoimmune and Inflammatory Disorders

(71) Applicant: P3 Innovations LLC, Cherry Hill, NJ (US)

(72) Inventor: Ramesh PAPPU, Cherry Hill, NJ (US)

(73) Assignee: P3 Innovations, LLC, Cherry Hill, NJ (US)

(21) Appl. No.: 15/175,692

(22) Filed: Jun. 7, 2016

Related U.S. Application Data
(60) Provisional application No. 62/238,904, filed on Oct. 8, 2015.

(51) Int. Cl. G01N 33/68; A61K 31/519; G01N 33/564; A61K 31/47

(52) U.S. Cl. CPC G01N 33/6866; A61K 31/47; A61K 31/519; G01N 33/564; G01N 33/6893; G01N 2333/56; G01N 2333/565; G01N 2333/57; G01N 2800/50; G01N 2800/7095

(57) ABSTRACT

The disclosure relates to methods and assay that assists in the diagnosis of autoimmune and chronic inflammatory disorders such as systemic lupus erythematosus and rheumatoid arthritis by analyzing drug-responsiveness of an interferon signal in a hematological sample (e.g., blood) obtained from a human subject. The assay involves comparing the interferon signal in a control aliquot of the sample with the same interferon sample in an aliquot that has been exposed to a therapeutic modality (e.g., combined with a drug) that is known to be efficacious to treat the disorder. A significant difference between the interferon signals of the control and treated aliquots that corresponds to a characteristic interferon signature for the disorder indicates that the subject is afflicted with, or is likely to develop, the disorder.

### CROSS-REFERENCES TO RELATED APPLICATIONS

This application is entitled to priority to U.S. provisional patent application No. 62/238,904 filed 8 Oct. 2015.

### BACKGROUND OF THE DISCLOSURE

The invention relates generally to the field of assessing autoimmune or chronic inflammatory disorders in human subjects.

Autoimmune and chronic inflammatory disorders (ACIDs) involve abnormal immune response of the body against substances and tissues that are normally and/or chronically present in the body that cause development of pathological symptoms. Such abnormal immune responses may be restricted to certain organs (as in autoimmune thyroiditis) or involve a tissues located at different body locations (e.g., Goodpasture's disease, which can adversely affect basement membrane in lung and kidney). A wide variety of ACIDs are known, and human subjects can be afflicted with more than one ACID simultaneously. Examples of relatively common ACIDs include systemic lupus erythematosus (SLE), rheumatoid diseases (RA), psoriatic arthritis (PA), and Sjorgen's syndrome (SS).

It is widely believed that many ACIDS exhibit an onset that substantially precedes clinically-observable symptoms or events in patients in whom such onset has occurred. In subjects who develop ACIDs, increased production of interferons (IFNs) is known to be associated with occurrence of clinical symptoms, and decreased IFN levels have been associated with efficacious treatment. By way of example, Banchereau et al. (2006, Immunity 25:383-392) observed overproduction of certain IFNs in patients diagnosed with SLE, and Ronnblum et al. (2013, Curr. Opin. Rheumatol. 25:248-253) reports IFN overproduction in patients afflicted with other autoimmune disorders.

IFNs are cytokines that are characteristically released from cells in response to the presence of a pathogen, such as a virus or a tumor cell. IFNs are widely recognized as exhibiting modulatory influences on immune responses, including responses involved in ACIDS, through interactions which occur between IFNs and cell surface receptors which specifically recognize them. Numerous individual human IFNs are known.

The various functions of IFNs and their interactions with cell surface receptors are diverse and widely described in the literature. An aspect of IFN function that is particularly relevant to this disclosure is that most or all ACIDs can be characterized by a pattern of expression of various IFNs in patients afflicted with ACIDS (i.e., an “IFN signal”), including the identities and relative amounts of the IFNs that are expressed. The identities and expression levels of IFNs can be detected in a variety of ways described in the art, including by detection of the IFN proteins themselves (e.g., in blood or other tissues) or by detection of mRNA encoding them in tissues in which they are produced.

Numerous therapeutic agents are known to be efficacious for treating patients afflicted with ACIDs, and at least some of the therapeutic effects of those agents are believed to be attributable to the ability of the agents to affect IFN production. For example, Bennett et al. (2003, J. Exp. Med. 197:711-723) observed that glucocorticoid treatment of SLE patients significantly decreased IFN production. However, the therapeutic and IFN-production-modulating effects of these agents have been observed only in patients who have already presented with clinical manifestations of ACIDs, including IFN overproduction. A significant disadvantage of current technologies relating to treatment of ACIDs is that clinical presentation of an ACID tends to occur well after the apparent onset of the ACID, when pathological effects attributable to IFN overproduction and other disease consequences have already occurred. Furthermore, the symptoms of different ACIDs can significantly resemble one another or symptoms of disorders other than ACIDs, particularly at early stages of ACID progression, and fluctuations in such symptoms can further confuse differential diagnosis.

It would be a significant advance if ACIDs could be detected substantially earlier, so that therapeutic or preventive interventions could be initiated farther (perhaps entirely) in advance of adverse ACID consequences. The ability to intervene therapeutically at an early phase in the pathogenesis of an ACID has the potential to prevent disease, to limit the loss of quality of life, and to limit costs to society. The subject matter described herein provides such an advance.

### BRIEF SUMMARY OF THE DISCLOSURE

The disclosure relates to a method for assessing the likelihood that a human subject who does not exhibit a clinically substantial symptom of an autoimmune or chronic inflammatory disease (ACID) will develop the ACID. The subject can, for example, be a subject having a risk factor for developing the ACID The method includes the steps of i) assessing an interferon signal that is characteristic of the ACID in both a control aliquot and a treated aliquot of a hematologic sample obtained from the subject and ii) comparing the interferon signals of the control and treated aliquots. The treated aliquot is an aliquot of the sample that has been combined with a drug that is effective to treat the ACID. The control aliquot is an aliquot of the same sample that has been treated substantially identically to the treated aliquot, except that it has not been combined with the drug. Suppression of an interferon signal characteristic of the ACID in the treated aliquot indicates that the subject is afflicted with or will develop the ACID. Similarly, greater suppression of the interferon signal characteristic of the ACID in the treated aliquot indicates a greater likelihood that the subject is afflicted with or will develop the ACID.

Examples of ACIDs which can be assessed in this manner include systemic lupus erythematosus (SLE), rheumatoid arthritis (RA), psoriatic arthritis (PA), and Sjorgren's syndrome (SS). If the ACID is SLE, the drug can be selected from the group consisting of corticosteroids, non-steroidal anti-inflammatory drugs, abatacept, tumor necrosis factor alpha inhibitors, anti-rheumatics, and combinations of these. If the ACID is RA, then the drug can beselected from the group consisting of corticosteroids, non-steroidal anti-inflammatory drugs, abatacept, tumor necrosis factor alpha inhibitors, anti-rheumatics, and combinations of these. If the ACID is PA, then the drug can be selected from the group consisting of corticosteroids, non-steroidal anti-inflammatory drugs, tumor necrosis factor alpha inhibitors, immune suppressants, anti-rheumatics, and combinations of these. If the ACID is SS, then the drug can be selected from the group consisting of hydroxychloroquine, methotrexate, and combinations of these.

The interferon signal that is assessed can be the expression level of at least one Type I interferon, such as one or more of interferon-alpha, -beta, and -gamma.

If the subject is assessed to have a substantial likelihood of developing the ACID, then one or more interventions can be performed to delay or prevent the ACID or to alleviate it.

### DETAILED DESCRIPTION

The disclosure relates to the field of diagnosing autoimmune and chronic inflammatory diseases (ACIDs), preferably in the preclinical state. Described are assays which facilitate early identification, monitoring, and intervention for human subjects who are afflicted with or who are developing, or likely to develop, an ACID. These assays involve assessing a hematological sample obtained from the subject for the presence of an IFN signal that is characteristic of the ACID and that changes to a state less characteristic of the ACID in response to the sample being subjected to a therapeutic intervention (e.g., a drug) that is effective for treating the ACID. Because both the ACID-specific IFN signal and the responsiveness of that IFN signal to the therapeutic intervention can be detected in advance of clinical presentation of symptoms, the assays described herein can be used to identify ACID patients at an early stage of disease development, permitting monitoring of ACID development and, potentially at least, mitigation, slowing, or even prevention of further ACID progression.

By way of example, early detection of an ACID in a human subject facilitates lifestyle changes (e.g., job choice or exposure to certain environments) which may reduce or eliminate exposure to stimuli which promote ACID progression. Further by way of example, early detection of an ACID facilitates early provision to the patient of pharmaceutical or other agents which will slow progression of the ACID, mitigate or reduce symptoms attributable to the ACID, or even reverse development of the ACID.

In the following sections of this disclosure, the general assay method is first described, after which various aspects and uses of the assay method are discussed.

### The Assay

The assay described in this section is useful for assessing the likelihood that a human subject who does not exhibit a clinically substantial symptom of an ACID will develop the ACID. The assay can also be used in patients who present clinically with an ACID to confirm the ACID with which the patient is afflicted (e.g., confirming or supplementing existing in vitro diagnostic testing techniques for ACID-afflicted patients).

Briefly summarized, the assay involves assessing an interferon (IFN) signal that is characteristic of the ACID in both i) a control aliquot and ii) a treated aliquot of a hematologic sample obtained from the subject (i.e., preferably two aliquots of the same sample, or two different hematological samples taken from the same patient). The treated aliquot is exposed to a therapeutic modality (e.g., combined with a drug) that is known to be effective to treat the ACID, and the control aliquot is not exposed to the therapeutic modality. Other than exposure to the therapeutic modality, the control and treated aliquots are preferably treated substantially identically.

The IFN signals from each of the control and treated aliquots are then compared. Suppression of the IFN signal in the treated aliquot, relative to the IFN signal in the control aliquot, is an indication (i.e., a suggestion, not necessarily a guarantee) that the subject from which the sample was obtained either a) is afflicted with the ACID or b) is developing (i.e., will develop) the ACID, regardless of whether symptoms of the ACID are clinically apparent. Suppression of the IFN signal in the treated aliquot manifests itself as the IFN signal of the treated aliquot being more nearly characteristic of the IFN signature of a human not afflicted with the ACID than the IFN signal of the control aliquot.

Put another way, if the IFN signal of the subject's sample is responsive to the drug, that indicates that the subject is afflicted, or is developing, the ACID. Correspondingly, greater suppression of the IFN signal in the treated aliquot indicates a greater likelihood that the subject will develop the ACID.

IFN signals and signatures are each characterized by the identities of the IFNs that are expressed and by the relative levels of expression of the expressed IFNs. A wide variety of methods have been described by others for assessing expression and expression levels of the various known IFNs, and it is not critical which method(s) of assessment are used in the assay described herein. By way of example, the identities and expression levels of IFNs can be assessed using a quantitative polymerase chain reaction (PCR) and reverse transcriptase (RT) based method for assessing IFN-encoding mRNA in a sample. Alternatively, immunological reagents specific for individual IFNs can be used to quantify IFN proteins in a sample. By way of example, microarray assays and ELISA kits are commonly used to detect IFNs (see, e.g., Mavragani et al., 2010, Arthritis Rheum. 62(2):392).

The identity of the hematologic sample used in the assay is not critical. Examples of suitable samples include whole blood, populations of isolated blood cells (e.g., populations of blood monocytes isolated by fluorescence-activated flow cytometry following labeling with fluorescently-labeled antibodies specific for certain monocytes), blood plasma, bone marrow, lymph, spleen, and thymus.

The control and treated aliquots of the sample are preferably generated by collecting a single hematological sample and dividing it between at least the control and treated aliquots (i.e., so that the sample-derived material in each aliquot is substantially identical). However, the subject-derived material in the control and treated aliquots can be gathered in discretely-collected samples obtained from the same subject, preferably closely in time to one another and preferably collected by the same method and from the same portion of the subject's body (e.g., venous blood).

In an alternative embodiment, a single aliquot of a hematological sample obtained from a subject is used both as the control aliquot and as the treated aliquot. This embodiment is performed by assessing the IFN signal in the aliquot prior to exposing the aliquot to the therapeutic modality (i.e., substantially identical to assessing the IFN signal in a control aliquot) and thereafter exposing the aliquot to the therapeutic modality and subsequently again assessing the IFN signal in the aliquot (i.e., akin to assessing the IFN signal in a treated aliquot discrete from the control aliquot). This embodiment has the potential drawback that it cannot distinguish IFN signal changes attributable to exposure to the therapeutic modality from IFN signal changes that would occur in the sample over time, regardless of exposure to the therapeutic modality. The embodiment may nonetheless be useful, for example, in situations in which only limited quantities of archived or stored sample material are available.

The purpose of comparing the control and treated aliquots of the subject's hematological sample is to observe changes in the IFN signal of the sample that are induced by the therapeutic modality (e.g., drug) to which the treated aliquot is exposed. In a subject who is afflicted with an ACID or who is developing an ACID, both the control aliquot and the treated aliquot of the hematological sample should initially (prior to exposure of the treated aliquot to the therapeutic modality) exhibit an IFN signal that is characteristic of the ACID. In the control sample, that IFN signal should persist during the period during which the treated aliquot is exposed to the therapeutic modality and its IFN signal is subsequently assessed. However, the IFN signal of the treated aliquot will be altered if the therapeutic modality to which it is exposed is a therapeutic modality that is efficacious to treat the ACID. Thus, by comparing the post-exposure IFN signal of the treated aliquot with the IFN signal of the control aliquot, one or more differences in IFN signal that are induced by exposure to the therapeutic modality can be discerned. Because the therapeutic efficacy of the therapeutic modality is known for one or more ACIDs, observation of a substantial difference in the IFN signals of the control aliquot and treated aliquot exposed to the therapeutic modality indicates that the subject is afflicted with one or more ACIDs for which the therapeutic modality exhibits therapeutic efficacy. This is particularly so when the difference(s) in the IFN signals are characteristic of the changes in IFN expression that would be expected upon efficacious treatment (i.e., mitigation) of the ACID.

Subjects who do not currently exhibit clinically-evident symptoms of an ACID, but who are nonetheless afflicted with the ACID or are developing the ACID, will exhibit an IFN signal that is characteristic of the ACID. However, many known and unknown factors affect IFN expression levels in humans, and it is not generally possible to define a “normal” or “non-diseased” level of expression for most IFNs in humans. Instead, it is understood that humans normally exhibit variable “background” IFN expression levels, even in the absence of disease.

Particularly at early (e.g., pre-clinical) stages of the ACID, an IFN signal characteristic of the ACID may not be readily discernible in view of variations in background IFN expression levels that normally occur in humans (whether or not afflicted with the ACID). For this reason, assessment of the IFN signal in a hematological sample alone (analogous to the control aliquot described herein) will often not be diagnostic of an ACID, particularly at early stages of ACID development, such as before noticeable clinical manifestation of the ACID. However, the assays described herein capitalize on changes in IFN signal that are induced by exposure of an ACID-afflicted human to a therapeutic modality that is known to be efficacious to treat the ACID. Thus, even though the existence of an ACID-specific IFN signal may be indistinguishable from normal IFN signal variation in an ACID-afflicted subject at early stages of ACID development (e.g., before the ACID is clinically detectable), the change in IFN signal attributable to exposure to the therapeutic modality will nonetheless be distinguishable from normal IFN signal variation in ACID-afflicted subjects. The assay described herein relies on detection of therapeutic-modality-associated change(s) in IFN signal. Such changes are observed as a difference between the IFN signal of the “control aliquot” (i.e., the aliquot that is not exposed to the therapeutic modality) of a human hematological sample described herein and the therapeutic-modality-exposed “treated aliquot” of the sample. Similarly, an increase over time in the difference between the IFN signals of control and treated aliquots obtained from the same patient is indicative that the patient is afflicted with, or is developing, the ACID.

Most or all ACIDs exhibit IFN signals that are characteristic of the ACID. At least some of these characteristic IFN signals are already known, and it is likely that other characteristic IFN signals (e.g., of ACIDs that are not yet fully characterized) will be identified in the future. The methods described herein can be used to discern IFN signals of any ACID having a characteristic IFN signal, regardless of whether that characteristic signal is known as of the date of this disclosure. The methods described herein can thus be applied both to ACIDs which have an already-known characteristic IFN signal and, in the future, to ACIDs for which characteristic IFN signals are subsequently understood.

IFN signals that are characteristic of humans afflicted with a individual ACIDs are known, and these characteristic IFN signals are sometimes referred to in the literature as “IFN signatures” of the corresponding ACIDs. By way of example, IFN signatures are known for ACIDs systemic lupus erythematosus SLE), rheumatoid arthritis (RA), psoriatic arthritis (PA), and Sjogren's syndrome (SS). The assays described herein seek to detect occurrence or development of an ACID in a human by detecting suppression of the IFN signature of the ACID. This is effected by identifying changes in the IFN signal of the treated aliquot, relative to the IFN signal of the control aliquot, that are the reverse of the IFN signature of the ACID. By way of example, the IFN signature of SLE includes over-expression of IFN-alpha and IFN-beta in humans afflicted with SLE; suppression of the IFN signature of SLE is thus manifested as a decrease in expression of IFN-alpha and IFN-beta in a treated aliquot exposed to a therapeutic modality that is efficacious for treating SLE, relative to expression of IFN-alpha and IFN-beta in a control sample (not exposed to the therapeutic modality) derived from the same hematological sample.

By way of example, SLE is characterized by IFN signals in which serum levels of IFN-alpha and IFN-gamma are elevated, relative to individuals not afflicted with SLE. Also, elevated serum IFN-alpha levels in SLE patients were also correlated with increased levels of serum immune complexes and inversely correlated with the number of peripheral lymphocytes in serum. Furthermore, SLE patients who exhibited erythema exhibited higher serum IFN-alpha and IFN-gamma levels than SLE patients who did not exhibit erythema (Kim et al., 1987, Clin. Exp. Immunol. 70:562-569). Therapeutic modalities which are known to be efficacious to treat SLE include drugs such as corticosteroids such as prednisone, non-steroidal anti-inflammatory drugs such as ibuprofen and naproxen sodium, anti-malarial drugs such as hydroxychloroquine, immune suppressants such as azathioprine, mycophenolic acid, leflunomide, methotrexate, and belimumab, and rituximab. Other known treatment modalities include whole body vibration treatment (performed on the treated aliquot), for example. Exposure of the treated aliquot to any of these therapeutic modalities (or to any SLE treatment modalities, whether presently known or hereafter discovered) can be expected to change the IFN signal of the treated aliquot in ways opposite the characteristic IFN signal of SLE.

RA is characterized by abnormal function(s) in Type I IFN pathways. By way of example, over-expression of IFN-beta, particularly in synovial membranes, is observed in RA, as are enhanced blood levels of type I IFNs (including IFNs-alpha and -beta). Type I IFN pathway abnormalities associated with RA are further described in Crow, 2010, Arthritis Res. 12(Suppl.1):S5 and Mavragani et al., 2010, Arthritis Rheum. 62(2):392, for example. Therapeutic modalities which are known to be efficacious to treat RA include drugs such as corticosteroids such as prednisone, non-steroidal anti-inflammatory drugs such as ibuprofen and naproxen sodium, abatacept, tumor necrosis factor alpha inhibitors such as etanercept, infliximab, adalimumab, golimumab, and certolizumab, and anti-rheumatics such as methotrexate, leflunomide, hydroxychloroquine, and sulfasalazine. Exposure of the treated aliquot to any of these therapeutic modalities can be expected to change the IFN signal of the treated aliquot in ways opposite the characteristic IFN signal of RA.

PA is characterized by abnormal function(s) in Type I IFN pathways. By way of example, over-expression of various type I IFNs (including IFN-gamma and various subtypes of IFN-alpha), particularly in psoriasitic lesions, is observed in PA patients. Type I IFN pathway abnormalities associated with PA are further described in Yao et al., 2008, PLOS One 3(7):e2737 and Peterson et al., 2006, Genes Autoimmunity 7:583-591. Therapeutic modalities which are known to be efficacious to treat PA include drugs such as corticosteroids such as prednisone, non-steroidal anti-inflammatory drugs such as ibuprofen and naproxen sodium, tumor necrosis factor alpha inhibitors such as etanercept, infliximab, adalimumab, golimumab, and certolizumab, immune suppressants such as azathioprine and cyclosporine, and anti-rheumatics such as methotrexate, leflunomide, and sulfasalazine. Exposure of the treated aliquot to any of these therapeutic modalities can be expected to change the IFN signal of the treated aliquot in ways opposite the characteristic IFN signal of PA.

SS is characterized by abnormal function(s) in IFN pathways. By way of example, over-expression of various type I IFNs (including IFNs-alpha, -geta, and -gamma). Type I IFN pathway abnormalities associated with SS are further described in Casciola-Rosne et al., 2015,

Arthritis Rheumatol. 67:2437-2446 and Nguyen et al., 2013, Frontiers Immunol. 4:142. Therapeutic modalities which are known to be efficacious to treat SS include drugs such as hydroxychloroquine and anti-rheumatics such as methotrexate. Exposure of the treated aliquot to any of these therapeutic modalities can be expected to change the IFN signal of the treated aliquot in ways opposite the characteristic IFN signal of SS.

The IFN signals of two samples are “compared,” as described herein, by analyzing at least the identity of relevant IFNs (including IFN subtypes, for example) that are expressed in the samples and the amount of each relevant IFN that is expressed. An IFN is “relevant” if expression of that IFN is known to be altered in humans afflicted with or developing an ACID that is being considered by the person comparing the IFN signals, relative to expression of the same IFN in a human not afflicted with or developing the ACID. For example, Banchereau (citation above) discloses that both IFN-alpha and IFN-beta are overproduced in humans afflicted with SLE. Thus, for an observer who wishes to detect whether or not a human subject is afflicted with or developing SLE, IFN-alpha and IFN-beta are both relevant IFNs to observe in the assays described herein. Using those assays, the observer would obtain a hematologic sample from the human, divide the sample into control and treated aliquots, expose the treated aliquot to a therapeutic modality that is efficacious for SLE, and assess IFN signals in both the control and treated aliquots. The IFN signals assessed for each of these aliquots would include both whether or not each of IFN-alpha and IFN-beta is expressed in each aliquot and, if so, how much IFN-alpha and/or IFN-beta is expressed in each aliquot.

No precise degree of difference in the IFN signals of control and treated aliquots derived from the same hematological sample is necessary to indicate that the subject is likely afflicted with, or is at risk of developing, the ACID. Nonetheless, the greater degree of suppression of the IFN signature characteristic of the ACID that is observed in the treated aliquot, the greater is the likelihood that the subject is afflicted with, or will likely develop, the ACID.

In one embodiment, a hematological sample obtained from a subject is divided into a control aliquot and multiple treated aliquots. Discrete treated aliquots can be exposed to different therapeutic modalities (e.g., different drugs, each of which is efficacious for treatment of the same or different ACIDs). The IFN signals of the discrete treated aliquots can be compared with the IFN signal of the control aliquot to provide information about multiple ACIDs, to provide multiple discrete analyses of the same ACID, or a combination of these. Thus, multiple assays for detecting existence or development of the same ACID, for detecting existence or development of discrete ACIDs, or a combination of these, can be performed simultaneously for a patient.

The precise method by which the treated aliquot is exposed to the therapeutic modality is not critical, so long as the therapeutic modality is able to exert its effect upon IFN-producing cells in the treated aliquot. Then the therapeutic modality is a drug, for example, a dose of the solid drug may be dissolved in the treated aliquot, or a solution or suspension of the drug in a fluid may be combined with the treated aliquot (the same fluid, less the drug, can also be combined with the control aliquot). Similarly, the amount of the therapeutic modality to which the treated aliquot is exposed is not critical, but should be an amount sufficient to exert a detectable degree of the therapy that is characteristic of the therapeutic modality upon the treated sample. By way of example, when the therapeutic modality is a drug and the treatment aliquot is a blood sample, the amount of the drug may be selected to match the ordinary blood concentration of the drug when it is used in therapy for the ACID.

Subjects from whom a hematological sample is collected and assayed as described herein can be selected from the general population at random. However, it is well known that ACIDs more commonly develop in populations groups having certain risk factors. Many IFN assessment methods involve significant expense, and population-wide performance of assays described herein for detecting ACIDs and their likely development can be uneconomical to perform on so wide a basis. It is therefore preferable to screen subjects for risk factors for ACIDs prior to performing the assays described herein. By way of example, individuals who are at high risk of developing ACIDs include first degree relatives of ACID patients. Other high risk populations include women and people of African, Hispanic, or Native American descent. It is recognized that a variety of other conditions, including a subject's vaccination history, a subject's occupational history, and environments and chemical agents to which a subject has been exposed can affect the likelihood that an individual will develop one or more ACIDs. The assays described herein can be performed on subjects who are selected based on these conditions. The assays described herein can also be used to identify other characteristics which increase a subject's likelihood of developing an ACID.

Once a subject has been identified as being afflicted with, or at risk for developing, an ACID (regardless of the existence of clinically substantial symptoms of the ACID), a variety of interventions can be undertaken. A therapeutic agent known to be effective to treat the ACID can be administered to the subject, for example, to delay or prevent onset of pathological symptoms of the ACID or to minimize or postpone the severity of such symptoms. The therapeutic agent can, but need not, be the same therapeutic modality used in the assay. If a preventative agent is known to be effective to decrease the likelihood of developing the ACID, then the preventative agent can be prophylactically administered to a human identified using the assays described herein as being likely to develop the ACID. Similarly, if a subject identified using the assays described herein as being likely to develop the ACID is exposed to environmental or work conditions which are known to increase the likelihood, rate of development, or severity of the ACID, then the subject can be shielded from (or advised to avoid) such conditions, based on the results of the assay.

The methods described herein are likely ineffective to predict or detect onset, or the likelihood of onset, of ACIDS which are not associated with alterations in IFN signaling. By way of example, some degenerative central nervous system disorders (e.g., Alzheimer's disease and amylotrophic lateral sclerosis) would be grouped by some investigators with other ACIDS, but these disorders are not known to be associated with altered IFN signaling. The methods described herein are expected to be non-functional for detection or prognostication of these disorders.

### Example

The subject matter of this disclosure is now described with reference to the following Example. This Example is provided for the purpose of illustration only, and the subject matter is not limited to this Example, but rather encompasses all variations which are evident as a result of the teaching provided herein.

### POC Study: Pre-Clinical In Vitro Diagnostic Testing for Autoimmune Diseases

The ability to intervene therapeutically at an early phase in the pathogenesis of a chronic autoimmune disease has the potential to prevent disease, limit the loss of quality of life and costs to society. At this very early stage of the disease, there are no clinical manifestations but an active autoimmune process. By understanding the known biologic mechanisms in autoimmune diseases and applying that knowledge to screen persons with a high risk of developing these diseases, the onset of these diseases can be prevented. At this time a number of novel treatments including targeted therapies have been approved for established clinical autoimmune diseases. However, their success in early established clinical autoimmune disease has not resulted in either a cure or prevention of progression of that disease. Therefore there is an unmet need to identify people at high risk early on in their preclinical state and to intervene appropriately in order to prevent the onset of autoimmune diseases. Individuals who are at high risk include first degree relatives with a genetic predisposition. Such individuals have a higher likelihood to develop autoimmune diseases and may benefit from such an intervention.

Briefly, in a majority of autoimmune diseases, one can assume that this preclinical stage includes a period of genetic risk, an exposure to environmental triggers, followed by a period of asymptomatic autoimmunity. That is followed by a stage of non-specific symptoms and eventually a well-defined disease state. There is emerging evidence about the genetic make of individuals, including genetic polymorphisms linked to known autoimmune diseases. Specific environmental factors leading to gene-environmental interactions can trigger biologic mechanisms that initiate this stage of preclinical autoimmunity. This period of autoimmunity is relatively benign as it does not produce clinical disease. However, there is a lag time between this state of benign autoimmunity and the onset clinical disease. This lag time has been well documented in certain autoimmune diseases such as systemic lupus erythematosus and rheumatoid arthritis.

Therefore an opportunity exits during the healthy part of an individual's life, to evaluate the future risk of developing an autoimmune disease. Over a period of time, these individuals at a high risk will pass through a benign phase of preclinical autoimmunity followed by clinical disease. Predict this process remains problematic. At this time, prediction rules and prediction models using statistical modeling in at-risk populations have been developed for certain autoimmune diseases. These models use multiple variables including genetic risk factors, family history, environmental triggers, occupational history, vaccination history and comorbidities. However, they have limitations in their ability to predict an individual's risk of developing autoimmune disease. There is a gap using current methodology and tools to predict the evolution of autoimmune disease in individuals at high risk and/or having a genetic predisposition.

Therefore we look at testing methods that help to screen for autoimmune diseases in a high risk population including first degree relatives with a genetic predisposition. Identifying these individuals, carefully following them over a period of time, and intervening with cost effective treatments are important steps towards primary prevention of autoimmune disease.

This method is intended to be used as a screening tool to identify individuals who are likely to develop autoimmune diseases. It is tested in individuals who have a high risk to develop autoimmune diseases that have established treatments.

In vitro diagnostic testing has the potential to benefit patients, providing information including differential diagnosis, identification of a patient subset, identification of potential responders to specific drugs, and ways to individualize therapy. Developing an in vitro diagnostic testing system can be complex. Certain key advantages of in-vitro testing include methods to identify a protein molecule of interest such as interferon or testing of new targeted therapies using methods such as high throughput screening. An important challenge is the difficulty in extrapolating these findings to the results of in vivo studies.

Oncology has in recent years led the research and application of such in vitro diagnostics testing with respect to cancer genes. This area of pharmacogenomics has been rapidly growing. Receptor hormones for estrogen were identified as valuable biomarkers for identifying patients for hormonal treatment in women with breast cancer including Her 2 Neu DNA protein and Epidermal Growth Factor Receptor protein. Enriching the patient population using in vitro diagnostics helps to identify patients most likely to respond to treatment, and is important for clinical decision making.

Taking this concept a step further, establishing therapeutic biomarkers including cancers genes has led to the understanding and the discovery of targeted drugs for cancer. At this time, a majority of known cancer genes are associated with a drug response and a majority of these drugs are associated with a single known cancer gene. These gene-drug interactions have generated complex molecular signatures leading to the discovery of biomarkers and related diagnostic assays.

Another important application of in vitro diagnostics is the preclinical burden assessment in cancer is the use of screening tests in a healthy population. A number of valid biomarkers have been developed to screen for cancer including prostate specific antigen (PSA) for prostate cancer.

Autoimmune diseases have similar challenges, including the identification of disease specific biomarkers, and their relationship to the genetic makeup of an individual in the preclinical phase. Currently these biomarkers are primarily autoantibodies, not protein molecular signatures such as interferon. These protein molecules may have a closer relationship to the genetic makeup of that individual than the autoantibodies.

The genetic makeup of high risk individuals including first degree relatives is an area that is developing rapidly in autoimmune diseases. Furthermore, we are making progress in understanding the relationship of a gene and a specific biomarker such as interferon in the preclinical phase of an autoimmune disease. An interferon signature can demonstrate a clinically meaningful response to known treatments for autoimmune diseases such as hydroxychloroquine or methotrexate when assessed using an in vitro diagnostic assay. This approach can identify “responders” in a high risk, but healthy, population who are likely to develop an autoimmune disease later in life. This novel approach of detecting biomarker- drug responsiveness in vitro is the foundation of the methods described herein.

### Proof of Concept Study

To establish the validity of such an in vitro testing system, a proof of concept study is a highly desirable step. The study involves a small number of subjects, including patients with established autoimmune diseases such as systemic lupus erythematosus, their first degree relatives, and age matched healthy controls. Whole blood as well as plasma samples are drawn from these subjects and analyzed for interferon levels and interferon gene signals using established methods. Drugs of interest, including hydroxychloroquine, are added to the whole blood or plasma in an amount that is determined using established parameters including optimal bioavailability, serum levels, and effective plasma/serum levels. Post mixing interferon levels are determined again using established methods. Drug responsiveness is determined by demonstrating a difference in the pre and post mixing levels of interferon. This difference is expected to be statistically significant between first degree relatives and the healthy control arm (null hypotheses). This target neutralization of interferon signature is the key element of the proposed diagnostic testing. Additional data are analyzed to observe any difference in the pre and post mixing levels of interferon between patients with established autoimmune disease and their first degree relatives.

The performance characteristic of an assay, its acceptable accuracy, precision, sensitivity, specificity as well as it positive predictive value are critical elements that can be established for such an assay using conventional methods. Pre specification of assay cutoffs, estimates of performance using statistical methods including receiver-operator characteristics are used to make such determinations. Clinical validity including clinical sensitivity and specificity help to determine patient population that are responders and non-responders. Statistical methods including likelihood ratios and confidence intervals are also applied.

In summary this proof of concept study is an analytical and clinical validation of an in vitro diagnostic test. This is an important step to establish its clinical utility to screen high risk patients with preclinical autoimmune diseases.

The disclosure of every patent, patent application, and publication cited herein is hereby incorporated herein by reference in its entirety.

While this subject matter has been disclosed with reference to specific embodiments, it is apparent that other embodiments and variations can be devised by others skilled in the art without departing from the true spirit and scope of the subject matter described herein. The appended claims include all such embodiments and equivalent variations.

What is claimed is:

1. A method for assessing the likelihood that a human subject who does not exhibit a clinically substantial symptom of an autoimmune or chronic inflammatory disease (ACID) will develop the ACID, the method comprising
assessing an interferon signal that is characteristic of the ACID in both
i) a control aliquot and
ii) a treated aliquot
of a hematologic sample obtained from the subject, wherein the treated aliquot is an aliquot of the sample that has been combined with a drug that is effective to treat the ACID and the control aliquot is an aliquot of the same sample that has been treated substantially identically to the treated aliquot, except that it has not been combined with the drug; and
comparing the interferon signals of the control and treated aliquots, whereby suppression of an interferon signal characteristic of the ACID in the treated aliquot indicates that the subject is afflicted with or will develop the ACID, and greater suppression of the interferon signal characteristic of the ACID in the treated aliquot indicates a greater likelihood that the subject is afflicted with or will develop the ACID.

2. The method of claim 1, wherein the ACID is selected from the group consisting of systemic lupus erythematosus (SLE), rheumatoid arthritis (RA), psoriatic arthritis (PA), and Sjorgren's syndrome (SS).

3. The method of claim 2, wherein the ACID is SLE and the drug is selected from the group consisting of corticosteroids, non-steroidal anti-inflammatory drugs, abatacept, tumor necrosis factor alpha inhibitors, anti-rheumatics, and combinations of these.

4. The method of claim 2, wherein the ACID is RA and the drug is selected from the group consisting of corticosteroids, non-steroidal anti-inflammatory drugs, abatacept, tumor necrosis factor alpha inhibitors, anti-rheumatics, and combinations of these.

5. The method of claim 2, wherein the ACID is PA and the drug is selected from the group consisting of corticosteroids, non-steroidal anti-inflammatory drugs, tumor necrosis factor alpha inhibitors, immune suppressants, anti-rheumatics, and combinations of these.

6. The method of claim 2, wherein the ACID is SS and the drug is selected from the group consisting of hydroxychloroquine, methotrexate, and combinations of these.

7. The method of claim 1, further comprising selecting a subject having a risk factor for developing the ACID.

8. The method of claim 7, wherein the risk factor is selected from the group consisting of genetic maker associated with predisposition for the ACID, a family history of occurrence of the ACID, a vaccination history associated with predisposition for the ACID, an occupational history associated with predisposition for the ACID, a history of exposure to an environment associated with predisposition for the ACID, occurrence of morbidity associated with predisposition for the ACID, and combinations of these.

9. The method of claim 1, wherein the interferon signal is assessed by determining the expression level of at least one Type I interferon.

10. The method of claim 9, wherein the interferon signal is assessed by determining the expression level of at least one interferon-alpha.

11. The method of claim 9, wherein the interferon signal is assessed by determining the expression level of at least one interferon-beta.

12. The method of claim 9, wherein the interferon signal is assessed by determining the expression level of at least one interferon-gamma.

13. The method of claim 1, wherein the treated aliquot of the sample is combined with an amount of the the drug that is sufficient to treat the ACID in a human subject afflicted with the ACID.

14. The method of claim 1, wherein the sample is whole blood.

15. A method of delaying the onset of an ACID in a human subject who does not exhibit a clinically substantial symptom of the ACID, the method comprising
assessing the likelihood that the subject will develop the ACID using the method of claim 1, and
administering to the subject an intervention known to delay onset of the ACID if the assessment indicates that the likelihood that the subject will develop the ACID is greater than a selected value.

16. A method of preventing an ACID in a human subject who does not exhibit a clinically substantial symptom of the ACID, the method comprising
assessing the likelihood that the subject will develop the ACID using the method of claim 1, and
administering to the subject an intervention known to prevent ACID if the assessment indicates that the likelihood that the subject will develop the ACID is greater than a selected value.

17. A method of delaying the onset of a selected symptom of an ACID in a human subject who does not exhibit a clinically substantial symptom of the ACID, the method comprising
assessing the likelihood that the subject will develop the ACID using the method of claim 1, and
administering to the subject an intervention known to delay onset of the symptom if the assessment indicates that the likelihood that the subject will develop the ACID is greater than a selected value

18. A method of treating an ACID in a human subject who does not exhibit a clinically substantial symptom of the ACID, the method comprising
assessing the likelihood that the subject will develop the ACID using the method of claim 1, and
administering to the subject an intervention known to treat the ACID if the assessment indicates that the likelihood that the subject will develop the ACID is greater than a selected value
